(19) United States
(12) Patent Application Publication
(10) Pub. No.: US 2025/0299596 A1
(43) Pub. Date: Sep. 25, 2025

(54) M.E.N.A's app (Music Engagement for Nonverbal Autism and Stuttering)

(71) Applicant: Anna Yael Blaine, Ocean, NJ (US)

(72) Inventor: Anna Yael Blaine, Ocean, NJ (US)

(21) Appl. No.: 18/616,083

(22) Filed: Mar. 25, 2024

(51) Int. Cl. G09B 19/04; A61B 5/00; G06F 3/04817; G10L 15/22

(52) U.S. Cl. CPC G09B 19/04; A61B 5/4803; G06F 3/04817; G10L 15/22

(57) ABSTRACT

M.E.N.A.'s app (Music Engagement for Nonverbal Autism and Stuttering) is an interactive, music-based speech therapy tech app, that uses Melodic Intonation Therapy in games to facilitate the production of speech in nonverbal children with autism and facilitate the fluency of speech in children who stutter. It will help nonverbal autistic children and children who stutter to communicate effectively in addition to helping them understand social cues and develop better social skills.

### FIELD OF INVENTION

M.E.N.A.'s app is an interactive speech therapy tech app that uses Melodic Intonation Therapy (MIT) in children's games to facilitate the production of speech and communication in nonverbal children with autism spectrum disorder (ASD) and children who have speech disfluency from stuttering.

### BACKGROUND OF THE INVENTION

Many speech language pathologists have used music and singing as a form of speech therapy for years. Melodic Intonation Therapy (MIT) is a technique that was developed in 1973 to help adults recovering from a stroke or brain injury that has impaired their ability to speak. In regards to stroke victims, this technique is used to treat Broca's aphasia which is severe damage to the left hemisphere of the brain where speech comes from. According to Melodic Intonation Therapy: Shared Insights on how it is done and why it might help (Norton, Zipse, Marchina and Schlaug), the original program is designed to lead nonfluent aphasic patients from intoning simple, 2-3 syllable phrases, to speaking 5+ syllable phrases 1, 3, 4 across 3 levels of treatment (2009).

Singing comes from the right hemisphere of the brain. MIT utilizes visual cues which act as a stimulus, melody, rhythm and intonation (singing) as a way of engaging the right hemisphere of the brain to learn how to speak again. Left hand tapping is used in MIT to synchronize syllable words and phrases to a melody. Furthermore, by asking a probe question during the session, this technique encourages response and auditory feedback to help in communication. Studies have shown that MIT can be effective in treating nonverbal children with ASD and improve speech fluency in children who stutter. In Critical Review: Beyond Adults with Aphasia, the Effectiveness of Intonation Based Therapy in Pediatric Populations (Riemersma), since MIT may have multiple mechanisms involved, it is reasonable to assume that it could be applied to a broader range of populations beyond adults with aphasia. Two such populations are children with autism and children with developmental apraxia of speech (2018), which can be found in children who stutter. This invention focuses on targeting the speech language difficulties of nonverbal children with autism and children who stutter to help improve communication and fluency using MIT, while also fusing MIT with interactive games for these children.

### SUMMARY OF THE INVENTION

M.E.N.A.'s app focuses on helping nonverbal children with autism and children who stutter to communicate effectively. The MIT interactive games will help these children with understanding social cues and building social skills.

### BRIEF DESCRIPTION OF THE DRAWINGS

The following descriptions are a brief summary of the drawings for this invention: FIG. 1—an illustration of the primary user interface which includes the user profile in addition to the ribbon, and the icons for the three levels of MIT.

FIG. 2—an illustration of the secondary user interface which includes the user profile in addition to the ribbon, the drop-down feature which collapses the icon when the level of MIT is selected and the arrow that points to the interactive MIT games session choices of musical chairs or telephone game.

FIG. 3—an illustration of the secondary user interface in addition to a question asking whether the interactive MIT games session is for nonverbal children or children who stutter.

FIG. 4a—an illustration of the third user interface in addition to a word bank with ten target words or phrases.

FIG. 4b—an illustration of the third user interface in addition to a word bank with ten target words or phrases.

FIG. 5—an illustration of the third user interface in addition to a video tutorial of an interactive MIT games session.

FIG. 6a—an illustration of the fourth user interface in addition to an icon that begins the MIT games session.

FIG. 6b—an illustration of the fourth user interface in addition to a visual cue of an apple followed by a melody, left hand tapping and a two-syllable word “APPLE.”

FIG. 6c—an illustration of the fourth user interface in addition to a visual cue of a mouth moving followed by a melody, left hand tapping and a six-syllable tongue twister phrase “TOM TUGGED THE TOY TUGBOAT.”

FIG. 6d—an illustration of the fourth user interface in addition to a visual cue of the game of musical chairs followed by a melody and left hand tapping.

FIG. 6e—an illustration of the fourth user interface in addition to a visual cue of the telephone game followed by a melody and left hand tapping.

FIG. 7—an illustration of the primary user interface in addition to the account settings and tech features that are included inside the ribbon.

### DETAILED DESCRIPTION OF THE INVENTION

The following description will explain the software design, the embodiments and the tech features of M.E.N.A.'s app. It will describe the various processes that this invention will perform to work as a music-based speech therapy tech app using MIT in the games of musical chairs and the telephone game.

In FIG. 1, the primary user interface is the main menu of the app. It is the first screen that will appear after the user (speech pathologist, teacher or parent) registers with an email and password. The primary user interface consists of the user profile and ribbon, but the main features of the first embodiment are the touch screen icons which represent the three levels of Melodic Intonation Therapy (MIT). This is where the user is introduced to the three levels of MIT:
1. E—elementary
2. I—intermediate
3. A—advanced.

Elementary is the basic level of MIT. Intermediate is a step above elementary level. Advanced is the master level. Think of it in terms of a puzzle. The better one gets at solving puzzles, the more they will increase the challenge from a fifty-word puzzle, to a one-hundred-word puzzle, to a thousand-word puzzle.

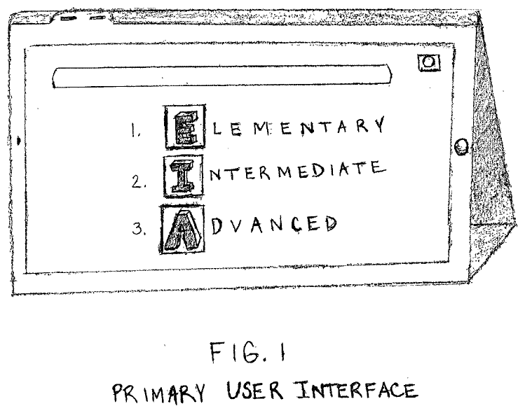

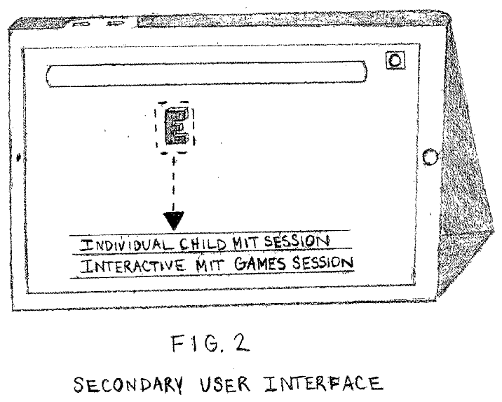

In FIG. 2, the secondary user interface appears when the user clicks on the icon of the level of MIT they select. As illustrated in FIG. 2, if a user clicks on the E-elementary icon, the drop-down feature will collapse the icon. This is the second embodiment of the app. The collapsed icon with the drop-down arrow will open up to two icons with an option for the user to select an interactive MIT games session of: musical chairs or the telephone game.

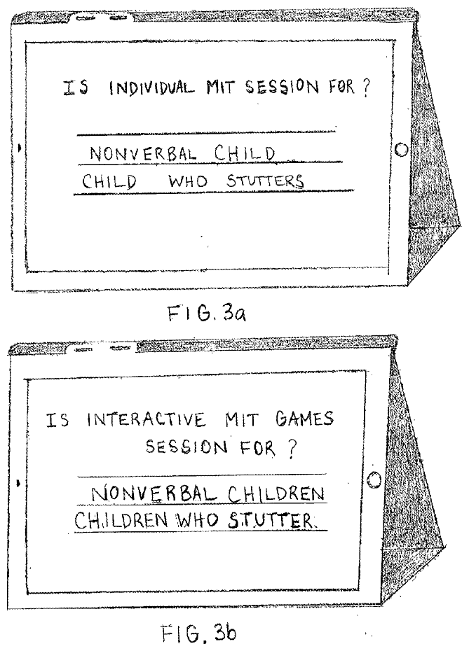

As illustrated in FIG. 3b, if the user selects an interactive MIT games session, a question will appear on the secondary user interface asking whether the games session is for nonverbal children with autism or children who stutter?

While the speech language difficulties of nonverbal children with autism and children who stutter has similarities, it also has differences. In an Inquiries Journal article, Vol. 10. No. 01, PG 1/1 entitled Benefits of Interactive Music Therapy on Children with Autism (Zhitnitsky), autism spectrum disorder is defined as a neurodevelopmental disorder with a wide range of severity, encompassing mild to severe levels of social, communicative, cognitive, and behavioral functioning (2018). According to a Q&A on Verbal Dyspraxia and Stuttering (Harmon Ph.D), stuttering can result from a variety of neurological disturbances and is not necessarily related to damage to any one structure or neural pathway (2004). Since autism and stuttering are different in how it is defined, the interactive MIT games session will be suited to those differences. This is why the games of musical chairs will preferably be suited to nonverbal children with autism and the telephone games will be preferably be suited to children who have a stutter. However, a speech pathologist, teacher or parent can play the game of musical chairs with children who stutter or play the telephone games for nonverbal children with autism. There are no set rules on this app that prohibit that.

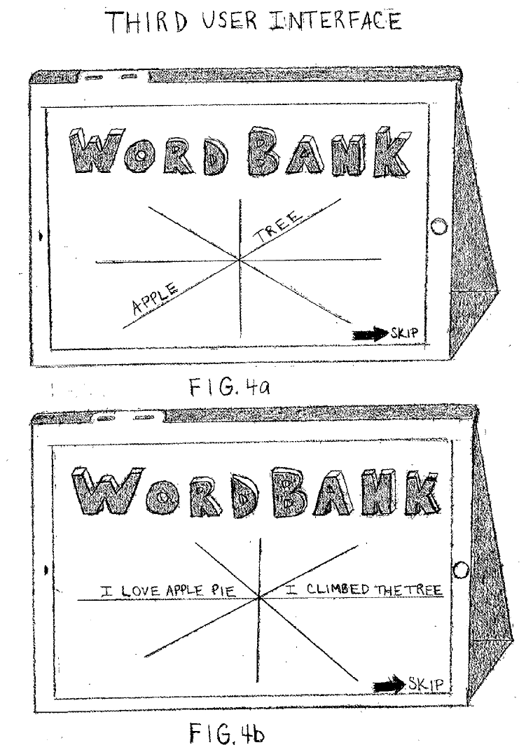

In FIG. 4a, the third user interface appears when the user has answered the questions that accompany the start of the interactive MIT games session. This is the third embodiment where syllable words or phrases are submitted into each session. As illustrated in FIG. 4a, a word bank is generated for the user to manually select ten target words or ten target phrases for a nonverbal child with autism. Examples of 1, 2 syllable words such as apple, tree, etc. FIG. 4b illustrates a word bank that is generated for the user to manually select ten target words or phrases for a child who stutters. Examples of 4, 5 syllable phrases such as I love apple pie, I climbed the tree, Tom tugged the toy boat. If the user so chooses, there is an option to skip over the word bank. If the user does skip over it, the app will automatically select target words and target phrases for each interactive MIT games session that is chosen. The word bank is designed to increase the target words and target phrases for intermediate and advanced levels of MIT. In the journal article, entitled Improving Pragmatics in Nonverbal Children with Autism Using Melodic Based Communication Therapy Vol. 3, Issue 3 (Sandiford, Mainess and Daher) Research indicates that the corpus callosum which joins both hemispheres and allows for transfer of information between hemispheres, is often impaired in children with autism (2013). Children with autism also experience sensory overload which means that they can have trouble processing lots of information that has to do with the five senses of taste, touch, sound, seeing and smelling. For this reason, autistic children may need more attention and help with melodic cues and intoning as opposed to children who stutter. The melodic cues and repetitive intonation will stimulate the area of the brain that is impaired in nonverbal children with autism.

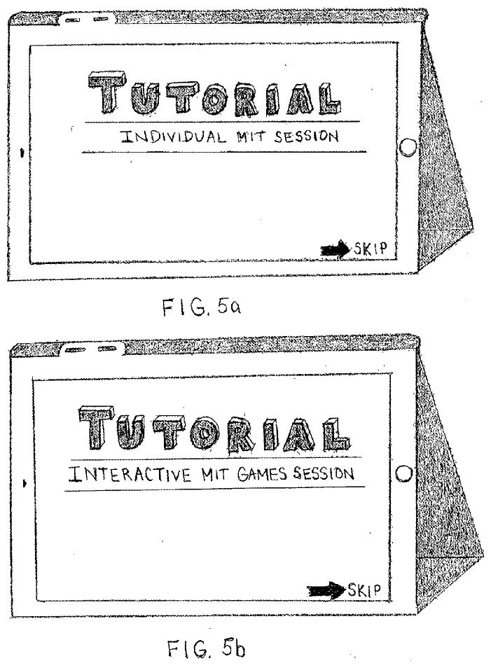

In FIG. 5, the third user interface shows a video tutorial of an interactive MIT games session for nonverbal children with autism or children who stutter. If the user so chooses, there is an option to skip over the tutorials. The tutorials aid the user on how to do MIT if they are not familiar with it.

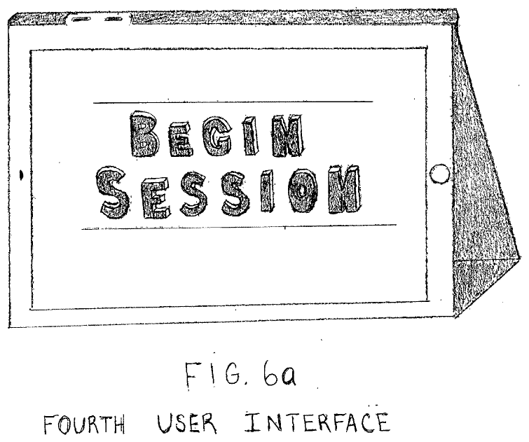

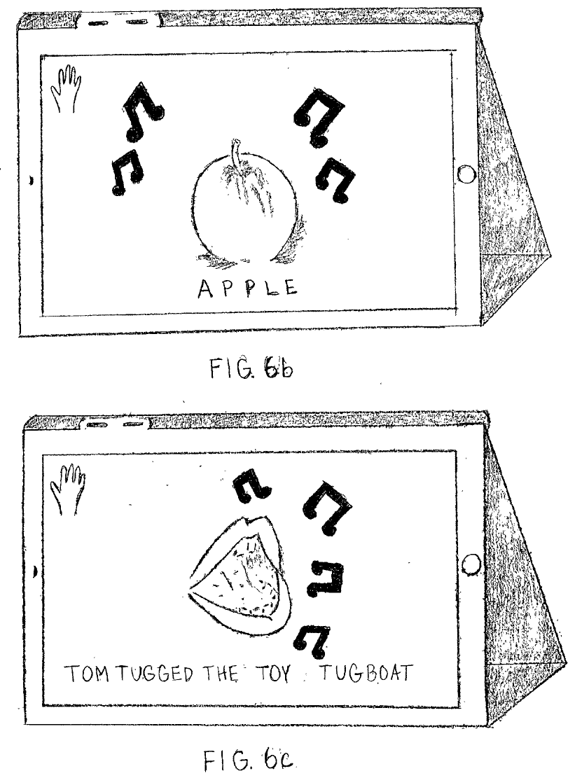

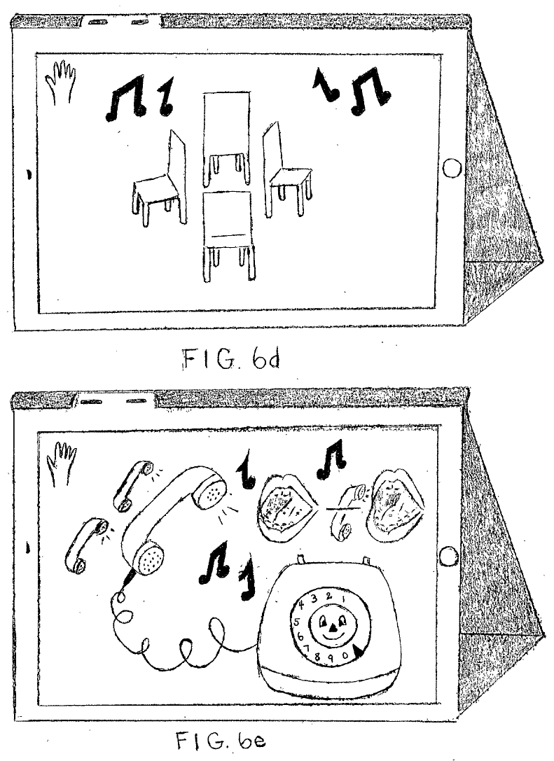

In FIG. 6a, the fourth user interface appears accompanied by a begin session icon. As illustrated in FIG. 6b, the interactive MIT games session of musical chairs for nonverbal children with autism begins with a visual cue of an apple, followed by a melody, the two-syllable word AP-PLE and the animated left hand tapping. This is the fourth embodiment of the app. Designed to synchronize the animated left hand tapping with a melody and guide the child into singing the syllable word or phrase to the melody. With the help of the user, asking a probe question to gauge the response of the child, this app will serve to function as a learning tool that encourages speech production, communication, auditory feedback and cognitive development. FIG. 6c illustrates an interactive MIT games session of the telephone game for children who have a stutter. The session begins with a visual cue of a moving mouth, followed by a melody, the six-syllable tongue twister phrase TOM TUGGED THE TOY TUG-BOAT and the animated left hand tapping. The moving mouth is to demonstrate how the child who stutters should relax their mouth and move their jaw when they are doing MIT.

FIG. 6d and FIG. 6e illustrates how MIT will be used in interactive games. The games will be limited to four children to keep it simple. In FIG. 6d, this is an illustrated example of the game of musical chairs for nonverbal children with autism. This is the fifth embodiment of the app. Designed to synchronize the animated left hand tapping with a melody and guide the children into singing the syllable word or phrase to the melody. Before each game, a visual cue of a syllable word or phrase will be shown on the app. Next, the user will hum along with the melody that synchronizes to the syllable word or phrase on the app before singing aloud. The four children will do an inner rehearsal before singing along in unison. The next visual cue that will be shown are the four chairs that will light up in a clockwise motion on the app which will be a cue for the children to march around chairs or in a circle. When the user fades out, the melody will stop playing and the children will sit down. The user will then ask the probe question. The three children that are the first to sing the answer which is the syllable word or phrase used in the game will go to the next round until there is one winner.

In FIG. 6e, this is an illustrated example of the telephone game for children who stutter. Before each game, a visual cue of a mouth moving followed by a melody and a syllable phrase, preferably a tongue twister phrase will be shown on the app. Next, the user will hum along with a melody that synchronizes to the syllable phrase on the app before singing aloud. The four children will do an inner rehearsal before singing along in unison. When the user fades out, the melody will stop playing. The next visual cue that will be shown are the four telephones that will light up as the syllable tongue twister phrase is sung in the ear of each child. When the user asks the probe question to the fourth child at the end, they must sing the phrase out loud correctly. If the child has no speech disfluency, the game is won and a new game can begin rotating the children. Speech pathologists, teachers or parents can feel free to do left hand tapping with the melody coming from the app if they so choose.

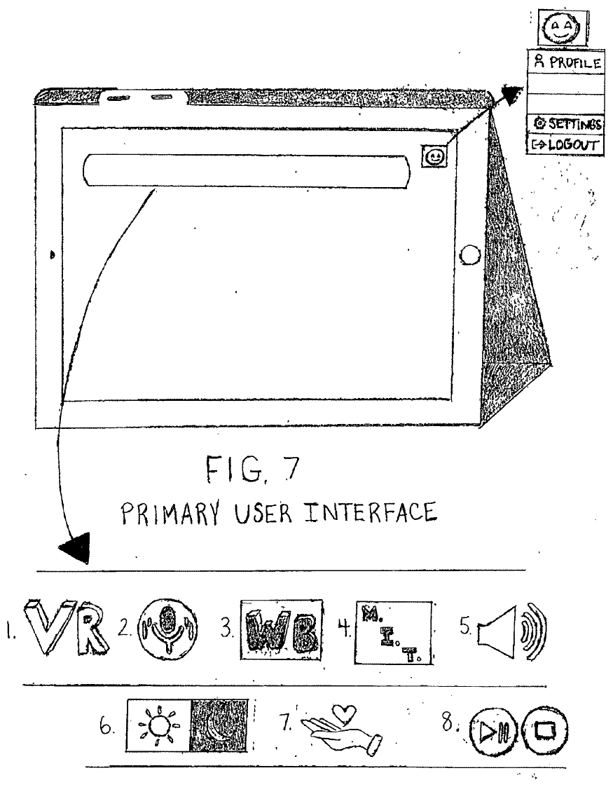

In FIG. 7, the app returns to the primary user interface which is the main menu. Here we have the entire layout of the user profile and the ribbon. The user profile is a tiny icon at the top right corner of the primary user interface. By clicking on the icon, a drop-down list appears. The account settings are located on that list. When the user clicks on the account settings, this is where the user's email address is stored and where the password can be updated. This is also where the user can upload an avatar for their profile picture.

As illustrated in FIG. 7, the ribbon is underneath the user profile. Inside the ribbon are widgets. This is the sixth embodiment which uses tech features to correctly recognize and respond to melodic speech, save recordings to examine progress, maintain help and support, and modify the visible settings of this app.
1. Voice Recognition is the first widget. This feature allows the app to recognize and pick up on the melodic speech of a child or children when they have answered the probe question correctly. Whenever the child or children answers correctly, a round of applause follows.
2. Recording and Playback is the second widget. This feature allows the app to record an interactive MIT games session. This way the speech pathologist, teacher or parent can watch and listen to a recording and play it back to see if the child or children are improving.
3. Word Bank is the third widget. This feature allows the app to keep track of what syllable words or phrases are used in each session. The user can go directly to the word bank to see what words and phrases were used if there is a particular word or phrase they want to reuse in another session.
4. MIT Tutorial is the fourth widget. This feature allows the user to watch a tutorial if they ever need to refresh their mind about how Melodic Intonation Therapy works.
5. Sounds and Haptics is the fifth widget. This feature allows the user to control the volume module and to activate Bluetooth so that the smartphone device or tablet that the app is on can pick up stronger signals.
6. Light Mode and Dark Mode is the sixth widget. This feature allows the user to switch to light mode where the screen and the icons are adjusted to a lighter setting. It also allows the user to switch to dark mode where the screen and the icons are adjusted to a darker setting.
7. Helping Hand is the seventh widget. This feature allows the app to give the child or children a helping hand if they are having trouble with the syllable words or phrases. The user can turn this feature on. Whenever a child is struggling with a syllable, the user can touch the syllable, the syllable will light up and an automated voice will intone the syllable for the child.
8. Start/Pause and Stop is the eighth widget. This feature allows the user to restart, pause or stop the interactive MIT games session.

1. A speech therapy app that uses Melodic Intonation therapy (MIT) in children's games to facilitate the production of speech in nonverbal children with autism and facilitate speech fluency in children who stutter.

2. A speech therapy app according to claim 1, wherein the user (teacher or parent) is directed to the primary user interface.

2. A speech therapy app according to claim 1, wherein the user (speech pathologist, teacher or parent) is directed to the primary user interface.

3. A speech therapy app according to claim 2, wherein the user interface consists of three separate icons which is the first embodiment.

4. A speech therapy app according to claim 3, wherein said icons represent the elementary level, the intermediate level and the advanced level of MIT.

5. A speech therapy app according to claim 4, wherein the user can click on the icon of the level of MIT they want to use in a session.

5. A speech therapy app according to claim 4, wherein the user can click on the icon of the level of MIT they want to use in a games session and said icon will collapse which is the second embodiment.

6. A speech therapy app according to claim 5, wherein the secondary user interface appears when the drop-down feature collapses the level of MIT that is selected which is the second embodiment.

6. A speech therapy app according to claim 5, wherein the secondary user interface appears after said icon collapses to show a drop-down feature where the user can select an interactive MIT games session of musical chairs or the telephone game.

7. (canceled)

8. A speech therapy app according to claim 1, wherein the user interface includes an option for the user to select if the child or children are nonverbal or a stutterer(s)

7. A speech therapy app according to claim 1, wherein the user interface includes an option for the user to select if the games will be for nonverbal children with autism or children who stutter.

9. A speech therapy app according to claim 1, wherein the third user interface appears which is the third embodiment, consisting of a word bank with ten target words/phrases for the nonverbal child with autism and twenty-five target words/phrases for the child who stutters.

8. A speech therapy app according to claim 7, wherein the third user interface appears which is the third embodiment, consisting of a word bank with ten target words or phrases for nonverbal children with autism and children who stutter.

10.-12 (canceled)

13.-23 (canceled)

24. A speech therapy app further comprising embodiments consisting of voice recognition, audio sound quality and a playback recording feature to analyze and examine the progress of each child.

19. A speech therapy app further comprising embodiments consisting of voice recognition, audio sound quality and a playback recording feature to analyze and examine the progress of each child.

25.-31 (canceled)

32-39 (canceled)

40. A speech therapy app that uses MIT in two interactive games that must have the same number of syllable words or phrases and must have the same number of four children for the game to be effective.

9. A speech therapy app that uses MIT in musical chairs and the telephone game further comprising the same number of syllable words or phrases and the same number of four children for the games to be effective.

41. A speech therapy app according to claim 39, wherein the fourth user interface appears which is the fourth embodiment and shows a visual cue of the game of musical chairs for nonverbal children with autism.

10. A speech therapy app according to claim 9, wherein the fourth user interface appears which is the fourth embodiment and begins the game of musical chairs with a visual cue of the syllable word or phrase selected from the word bank for nonverbal children with autism.

42. (canceled)

43. A speech therapy app according to claim 40, wherein a melody will play and the four chairs will light up one-by-one in a counterclockwise motion on the app.

16. A speech therapy app according to claim 15, wherein the children will sing in unison until the user slowly fades out leaving said children to continue singing while a new visual cue of the game of musical chairs will show four chairs lighting up in different colors one-by-one in a clockwise motion on the app.

44. A speech therapy app according to claim 43, wherein the user will begin to hum along with the melody.

11. A speech therapy app according to claim 10, wherein the user will begin to hum along with the melody.

45. A speech therapy app according to claim 44, wherein the user interface will have a small, left hand in the corner of the screen that includes animated tapping.

12. A speech therapy app according to claim 11, wherein the game of musical chairs will have a small, left hand in the corner of the screen that includes animated tapping.

46. A speech therapy app according to claim 45, further comprising the animated left hand tapping to synchronize to the melody.

13. A speech therapy app according to claim 12, further comprising the animated left hand tapping to synchronize to the melody.

47. A speech therapy app according to claim 46, wherein the user will begin to intone (sing) the syllable word or phrase.

14. A speech therapy app according to claim 13, wherein the user will begin to intone (sing) the syllable word or phrase in said visual cue to the melody in the game of musical chairs.

48. A speech therapy app according to claim 47, wherein the melody and user will guide said children to do inner rehearsal preparing them to sing the syllable word or phrase.

15. A speech therapy app according to claim 14, wherein the melody and user will guide the children to do an inner rehearsal preparing them to sing the syllable word or phrase.

49. (canceled)

50. A speech therapy app according to claim 49, wherein the melody stops and said children will sit down.

17. A speech therapy app according to claim 16, wherein the melody stops and said children will sit down

51. A speech therapy app according to claim 50, wherein the user will ask said children a probe question.

18. A speech therapy app according to claim 17 wherein the user will ask said children a probe question.

52. A speech therapy app according to claim 24, wherein the user interface will include a round of applause in response to the voice recognition after said three children sing the syllable word or phrase correctly.

20. A speech therapy app according to claim 19, wherein the game of musical chairs will include a round of applause in response to the voice recognition after the first three children sing the syllable word or phrase correctly.

53. A speech therapy app according to claim 52, wherein another round of applause will happen after said children play the game of musical chairs to a new syllable word or phrase for each round until they reach the bottom two children and one child is declared the winner.

21. A speech therapy app according to claim 20, wherein another round of applause will happen after said children play the game of musical chairs to a new syllable word or phrase for each round until, they reach the bottom two children and one child is declared the winner.

54. (canceled)

55. A speech therapy app according to claim 54, wherein the fourth user interface appears which is the fourth embodiment and shows a visual cue of the telephone game for children who stutter.

22. A speech therapy app according to claim 9, wherein the fourth user interface appears which is the fourth embodiment and begins the telephone game with a visual cue of relaxed mouth movements, followed by a tongue twister syllable phrase selected from the word bank for children who stutter.

56.-57 (canceled)

58. A speech therapy app according to claim 57, wherein the user will begin to hum along with the melody.

23. A speech therapy app according to claim 22, wherein the user will begin to hum along with the melody.

59. A speech therapy app according to claim 58, wherein the user interface will have a small, left hand in the corner of the screen that includes animated tapping.

24. A speech therapy app according to claim 23, wherein the telephone game will have a small, left hand in the corner of the screen that includes animated tapping.

60. A speech therapy app according to claim 59, further comprising the animated left hand tapping to synchronize to the melody.

25. A speech therapy app according to claim 24, further comprising the animated left hand tapping to synchronize to the melody.

61. A speech therapy app according to claim 60, wherein the user will begin to intone (sing) to the phrase.

26. A speech therapy app according to claim 25, wherein the user will begin to intone (sing) the tongue twister syllable phrase to the melody while said visual cue mimics the user's mouth movements.

62. A speech therapy app according to claim 61, wherein the melody and user will guide the children to do inner rehearsal preparing them to sing the phrase in each other's ear.

27. A speech therapy app according to claim 26, wherein the melody and user will guide the children to do inner rehearsal preparing them to sing the phrase in each other's ear.

63. A speech therapy app according to claim 62, wherein the children will sing in unison until the user slowly fades out leaving said children to sing the phrase in each other's ear while simultaneously the telephones light up one-by-one on the app.

28. A speech therapy app according to claim 27, wherein the children will sing in unison until the user slowly fades out leaving said children to sing the phrase in each other's ear while a new visual cue of four telephones will light up in different colors one-by-one on the app.

64. A speech therapy app according to claim 63, wherein the melody stops and the user will ask a probe question to the last child in the telephone line.

29. A speech therapy app according to claim 28, wherein the melody stops and the user will ask a probe question to the last child in the telephone line.

65. A speech therapy app according claim 24, wherein the user interface will include a round of applause in response to the voice recognition after the last child at the end of the telephone line in the game sings the phrase fluently.

30. A speech therapy app according claim 19, wherein the telephone game will include a round of applause in response to the voice recognition after the last child at the end of the telephone line in the game sings said tongue twister phrase fluently.
